(19) United States
(12) Patent Application Publication
(10) Pub. No.: US 2024/0225812 A1
(43) Pub. Date: Jul. 11, 2024

(54) MEDICAL DEVICE

(71) Applicant: SHANGHAI BLUEVASCULAR MEDTECH CO., LTD., Shanghai (CN)

(72) Inventors: Zhaoduo ZHANG, Shanghai (CN); Baisi LIAN, Shanghai (CN); Hongbo WANG, Shanghai (CN); Zhichao REN, Shanghai (CN); Liwen WANG, Shanghai (CN); Zhenyu YUAN, Shanghai (CN); Zhenghua MIAO, Shanghai (CN)

(21) Appl. No.: 18/559,861

(22) PCT Filed: May 18, 2022
(86) PCT No.: PCT/CN2022/093664

§ 371 Date: Nov. 9, 2023

(30) Foreign Application Priority Data
Jun. 4, 2021 (CN) 202110625886.9

(51) Int. Cl. A61F 2/01

(52) U.S. Cl. CPC A61F 2/011; A61F 2/0105; A61F 2220/0016; A61F 2250/001

(57) ABSTRACT

The present invention provides a medical device including a delivery mechanism and an implant, wherein the delivery mechanism includes a delivery component and an assistive component that is connected to the delivery component and configured to be expanded or shrunk in a radial direction of the delivery component, and the implant is detachably connected to the delivery component; the delivery mechanism is configured to deliver the implant to a predetermined position in a target lumen and release the implant, and the assistive component is radially expanded during the release of the implant so as to release the implant with a predetermined form in the predetermined position. The advantage of the medical device is that the implant is positioned with a predetermined form in the target lumen, improving the efficiency for thrombus interception and facilitating subsequent retrieval.

### TECHNICAL FIELD

The present invention relates to the technical field of medical instrument and, more specifically, to a medical device.

### BACKGROUND

Venous thromboembolism (VTE) is a common clinical disease, with a high incidence and case fatality rate. The VTE includes deep vein thrombosis (DVT) and pulmonary embolism (PE). The DVT usually occurs in the lower limb veins, and the PE mainly results from the thrombosis which are formed in the venous system or right heart and then fall off into the pulmonary artery. This is the main cause of disease and death.

Anticoagulation therapy has always been the gold standard for treating VTE, which aims to prevent a formation of thrombosis, prevent PE, and restore the patency of the embolized veins. In the case that a patient has a contraindication to anticoagulation or has blood complications and thus has to terminate the anticoagulation, it is possible to implant a vena cava filter (VCF) to intercept the fallen thrombosis so as to prevent the occurrence of fatal PE. Existing filters are prone to tilt when implanted into the inferior vena cava and do not achieve the expected effect in thrombus interception. In addition, existing filters are also prone to shift after implantation into the inferior vena cava, which is not conducive to subsequent retrieval of the filters.

### SUMMARY OF THE INVENTION

The object of the present invention is to provide a medical device by which an implant can be released with a predetermined form in a target lumen, thereby improving the effect in thrombus interception, and thus the therapeutic effect.

To achieve the above object, the present invention provides a medical device, comprising a delivery mechanism and an implant, wherein the delivery mechanism comprises a delivery component and an assistive component that is connected to the delivery component and configured to be expanded or shrunk in a radial direction of the delivery component, and the implant is detachably connected to the delivery component;

wherein the delivery mechanism is configured to deliver the implant to a predetermined position in a target lumen and release the implant, and the assistive component is radially expanded during the release of the implant so as to release the implant with a predetermined form in the predetermined position.

Optionally, the delivery component comprises a tube assembly and a backward release component, the tube assembly comprising at least a first tube, the backward release component having a distal structure that is partially connected to the first tube and coaxially to the first tube, the assistive component sleeved over the first tube and proximal to the distal structure, the implant detachably connected to the distal structure of the backward release component.

Optionally, the assistive component comprises a first transition section, a main section and a second transition section which are axially connected successively, and an outer diameter of the first transition section and an outer diameter of the second transition section decrease in directions away from the main section;

wherein the assistive component is configured to at least partially movable in an axial direction of the first tube to radially expand or shrink the assistive component.

Optionally, an outer diameter of at least some segments of the main section is greater than or equal to a maximum outer diameter of the first transition section and the second transition section.

Optionally, the tube assembly further comprises a second tube that is sleeved over the first body and axially movable relative to the first tube and the distal structure;

wherein the assistive component has a distal end remained stationary relative to the first tube, and a proximal end that is connected to a distal end of the second tube and movable synchronously with the second tube.

Optionally, the delivery component further comprises a handle provided with a first drive assembly that is connected to a proximal end of the second tube and configured to drive the second tube to move axially relative to the first tube.

Optionally, the first transition section and the second transition section are symmetrically disposed at two axial ends of the main section; or wherein a length of the first transition section is less than a length of the second transition section, and the first transition section is closer to the implant.

Optionally, the assistive component is a balloon that is connected to and in communication with a perfusion channel defined in the first tube, and the perfusion channel is configured to fill an agent into the balloon.

Optionally, the backward release component comprises a limit sleeve, a restraint member and a connector, wherein the limit sleeve and the restraint member constitute the distal structure of the backward release component, the limit sleeve is connected to and in communication with the first tube, the limit sleeve is axially stationary relative to the first tube; the restraint member is configured to be received in the limit sleeve; the connector is inserted into the the first tube and movable relative to the first tube, and a distal end of the connector is connected with the restraint member;

wherein the restraint member is engaged with the limit sleeve and detachably connected to the implant when the restraint member is at least partially located inside the limit sleeve, and the backward release component is disconnected from the implant when the restraint member is at least partially exposed from a distal end of the limit sleeve and the restraint member is disengaged with the limit sleeve, resulting from a movement of the first tube relative to the connector.

Optionally, the delivery component further comprises a handle provided with a second drive assembly, the handle connected to a proximal end of the connector, the second drive assembly connected to a proximal end of the first tube and configured to drive the first tube to move axially relative to the connector.

Optionally, the implant comprises a plurality of filter rod groups and a retrieval part, each of the filter rod groups comprising a plurality of filter rods, proximal-to-heart ends of the filter rods in all the filter rod groups connected to the retrieval part; wherein the filter rods in different ones of the filter rod groups have different lengths, and all the filter rods in a same one of the filter rod groups have a same length and are arranged symmetrically around an axis of the implant;
wherein the retrieval part is configured to detachably connect to the backward release component; or
the medical device further comprises a sheath, shortest ones of the filter rods configured to be compressed in the sheath, distal-to-heart ends of rest of the filter rods configured to be inserted into the limit sleeve, distal-to-heart ends of longest ones of the filter rods configured to be connected to the restraint member and the limit sleeve.

Optionally, the restraint member is a slider having a side wall defined therein with a groove;

wherein the groove and an inner surface of the limit sleeve together limit a position of a proximal-to-heart end or a distal-to-heart end of the implant so as to connect the implant with the backward release component when the slider is at least partially received in the limit sleeve, and the backward release component is disconnected from the implant when the slider moves axially relative to the limit sleeve to expose the groove from the limit sleeve.

Optionally, the restraint member is an elastic member; when the elastic member is received in the limit sleeve, the limit sleeve applies a radial pressure to the elastic member to shape the elastic member as a hook for hooking the implant; when the elastic member at least partially protrudes from the limit sleeve, the radial pressure applied by the limit sleeve to the elastic member is removed, and the elastic member returns to a form that does not have the hook to release the implant.

Compared with the prior art, the medical device according to the present invention has the following advantages.

The medical device includes a delivery mechanism and an implant. The delivery mechanism includes a delivery component and an assistive component that is connected to the delivery component and configured to be expanded or shrunk in a radial direction of the delivery component. The implant is detachably connected to the delivery component. The delivery mechanism is configured to deliver the implant to a predetermined position in a target lumen and release the implant, and the assistive component is radially expanded during the release of the implant so that the implant is released with a predetermined form in the predetermined position. This avoids a decreased efficiency for thrombus interception resulting from a deviation of the implant from the predetermined form, thereby improving therapeutic effect.

Furthermore, the implant can be released by the backward release component step by step so as to prevent the implant from a displacement resulting in a deviation from a predetermined position during the release thereof. Besides, at least some of the filter rods of the implant is provided with the anchor for penetrating the inner wall of the target lumen so that the implant can be maintained at the predetermined position. The puncture-proof member is also provided for limiting the depth at which the anchor can penetrate the inner wall of the target lumen, thereby preventing the anchor from puncturing the inner wall of the target lumen and thus avoiding a damage to it.

### BRIEF DESCRIPTION OF THE DRAWINGS

The drawings are used for a better understanding of the present invention, and do not limit the present invention in any case.

FIG. 1 is a schematic diagram showing a structure of the medical device according to an embodiment of the present invention, where a filter is not shown;

FIG. 2 is a schematic diagram showing a structure of a delivery mechanism of the medical device according to an embodiment of the present invention;

FIG. 3 is a schematic diagram showing a structure of a filter of the medical device according to an embodiment of the present invention;

FIG. 4 is a schematic diagram of the medical device in use according to an embodiment of the present invention, showing a delivery mechanism delivering a filter to the inferior vena cava via the jugular vein;

FIG. 5 is a schematic diagram showing the connection between a backward release component and a filter in the medical device according to an embodiment of the present invention;

FIG. 6 is a schematic diagram showing the connection between the backward release component and the filter in the medical device according to another embodiment of the present invention;

FIG. 7 is a schematic diagram showing a structure of an expander according to another embodiment of the present invention;

FIG. 8 is a schematic diagram showing a structure of a sheath according to another embodiment of the present invention;

FIG. 9 is a schematic diagram of the medical device in use according to an embodiment of the present invention, showing a delivery mechanism delivering a filter to the inferior vena cava via the jugular vein;

FIG. 10 is a schematic diagram showing the connection between a backward release component and a filter in the medical device according to an embodiment of the present invention.

### LIST OF REFERENCE SIGNS

1000: delivery mechanism;
1100: delivery component;
1110: tube assembly;
1111: first tube, 1112: second tube;
1121: limit sleeve, 1122: restraint member;
1130: handle;
1131: slot, 1142: manipulator;
1140: first drive assembly;
1141: first slider;
1151: knob;
1200: assistive component;
1210: first transition section, 1220: main section, 1230: second transition
section;
1300: storage tube;
1400: expander;
1410: radiographic hole, 1420: second visualization element;
1500: sheath;
1510: first visualization element;
2000: filter;
2100: retrieval part;
2210: filter rod, 2210a: first filter rod, 2211a: first part, 2212a: second part, 2213a: third part, 2210b: second filter rod, 2210c: third filter rod, 2210d: fourth filter rod, 2310: first anchor, 2320: second anchor, 2410: puncture-proof member.

### DETAILED DESCRIPTION

The embodiments of the invention are described by specific examples, and those skilled in the art can easily understand other advantages and functions of the invention based on the contents disclosed in the specification. The invention may be implemented or applied based on different embodiments, and the details in the specification may be modified or changed without the departure from the spirit of the invention. It should be noted that the figures provided in the present embodiment only illustrates the basic idea of the present invention, showing only the components related to the present invention rather than the number, shape and size of the components in actual use. The configuration, number and proportion of the components in actual use can be optionally modified, and the arrangements of the components may be more complex.

Further, the embodiments described in the following each have one or more technical features, but this does not mean that all the technical features of the invention shall be implanted together, or only some or all of the different embodiments shall be be implemented separately. In other words, on the basis that the implementation is possible, a person skilled in the art may selectively implement, according to design standards or actual needs, some or all of the technical features in any embodiment, or some or all of the technical features of the multiple embodiments, so as to increase the flexibility of the present invention.

As used in this specification, the singular forms “a/an”, “one” and “this” include plural objects, and the plural form includes more than two objects, unless otherwise explicitly specified. As used in this specification, the term “or” is usually used to include the meaning of “and/or”, unless otherwise clearly specified, and the terms “installation”, “connection”, “coupling” shall be understood generally, for example, as a fixed connection, a detachable connection, or an integrated connection. It can be either a mechanical connection or an electrical connection. It can be a direct connection or an indirect connection through using an intermediate medium, and it can be an intercommunication between two elements or an interaction between two elements. For those skilled in the art, the specific meaning of the above terms in the present invention can be understood under specific circumstances.

To make the purposes, advantages and characteristics of the present invention clear, the present invention will be further described in detail in conjunction with the drawings. It should be noted that the drawings are in very simplified form and not made to scale, and only intent to facilitate a clear illustration for embodiments of the invention. The same or similar reference signs in the drawings represent the same or similar parts.

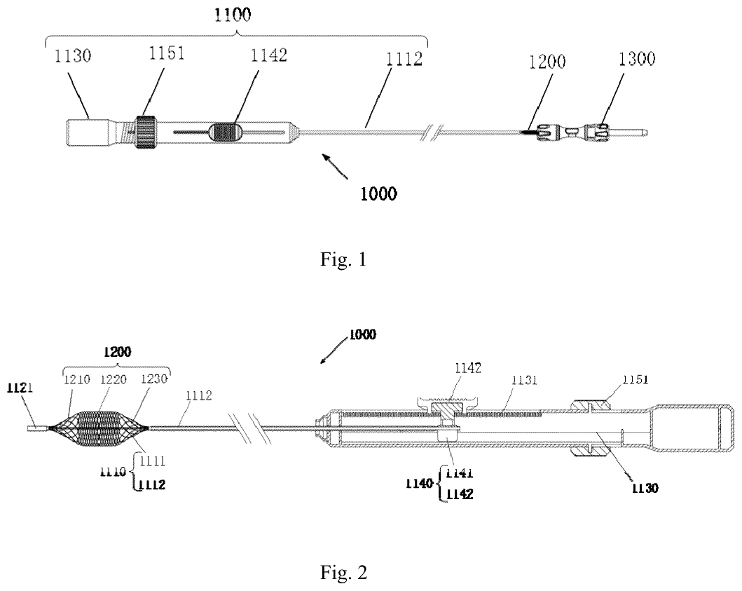

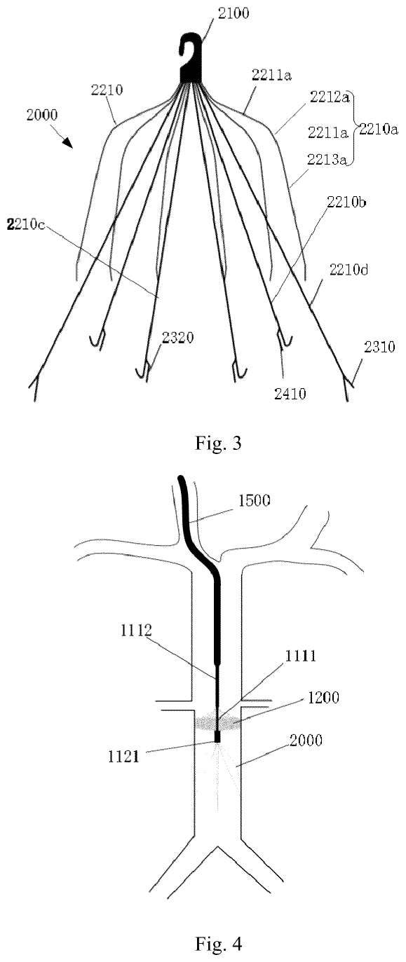

FIG. 1 is a schematic diagram showing a structure of the medical device; FIG. 2 is a schematic diagram showing a structure of a delivery mechanism 1000 of the medical device; FIG. 3 is a schematic diagram showing a structure of a filter 2000 of the medical device.

Referring to FIGS. 1 to 3, the medical device includes a delivery mechanism 1000 and an implant, for example, a filter 2000. The delivery mechanism 1000 includes a delivery component 1100 and an assistive component 1200 that is connected to the delivery component 1100 and configured to expand or shrink in a radial direction of the delivery component 1100; the filter 2000 is detachably connected to the delivery component 1100. The delivery mechanism 1000 is configured to deliver the filter 2000 to a predetermined position in a target lumen and release it at the position. During the release of the filter 2000, the assistive component 1200 radially expands in the radial direction of the delivery component 1100 and the filter 2000 is released with a predetermined form at the predetermined position. Generally, the filter 2000 may be implanted into the inferior vena cava to intercept the thrombus and prevent it from entering the heart and then into the pulmonary artery and causing pulmonary embolism. In practice, when the filter 2000 is implanted into the inferior vena cava, it is desired to position filter 2000 centrally in the inferior vena cava to improve the thrombus capture effect. The term “centrally” means that the filter 2000 is arranged coaxially with the inferior vena cava. In this case, the target lumen is the inferior vena cava, and the predetermined form of the filter 2000 may refer to that in which the filter 2000 can be coaxial with the inferior vena cava. In an embodiment of the present invention, the assistive component 1200 is connected to the delivery component 1100, and the assistive component 1200 is configured to assist the filter 2000 for release so that the filter 2000 can be released with a predetermined form (i.e., coaxially) in the inferior vena cava, thereby improving the interception effect of the filter 2000 on the thrombus and improving the therapeutic effect. It is understood that after the release of the filter 2000, the delivery component 1100 is withdrawn together with the assistive component 1200.

In detail, with reference to FIG. 2, in some embodiments, the assistive component 1200 preferably has a tubular mesh structure and includes a first transition section 1210, a main section 1220 and a second transition section 1230 which are axially connected successively, and the outer diameter of the first transition section 1210 and the outer diameter of the second transition section 1230 decrease in directions away from the main section 1220. The assistive component 1200, the delivery component 1100, and the filter 2000 are arranged coaxially. After the delivery component 1100 delivers the assistive component 1200 and the filter 2000 to a predetermined position in the inferior vena cava, the filter 2000 may be released. During the release of the filter 2000 (i.e., when the filter 2000 is not fully released), the assistive component 1200 expands radially, at least part of the outer wall of the main section 1220 of the assistive component 1200 is in contact with the internal wall of the inferior vena cava, the main section 1220 is then supported onto the inner wall of the inferior vena cava, the assistive component 1200 is coaxial with the inferior vena cava, the filter 2000 is thus coaxial with the inferior vena cava. The assistive component 1200 in the embodiment of the present invention is preferably a self-expanding structure which does not block blood flow when expanding in the inferior vena cava and supported on the vessel wall. Those skilled in the art know that the self-expanding structure is made from a material with high elasticity, and may deform when subjected to external pressure, and once the external pressure is cancelled, it will return to its initial form by virtue of the high elasticity. Generally, the material of the self-expanding structure can be a shape memory material such as nickel-titanium alloy. The assistive component 1200 may be formed by wire weaving or by tube cutting.

In this embodiment, the outer diameter of at least part of the main section 1220 is greater than or equal to the maximum outer diameter of the first transition section 1210 and the second transition section 1230. Preferably, the main section 1220 includes a plurality of cylindrical segments, each having an outer diameter equal to the maximum outer diameter of the first transition section 1210 and the second transition section 1230. The cylindrical segments are configured to contact with the wall of the inferior vena cava so that the main section 1220 is in surface contact with the inner vessel wall, which allows a large contact area and stronger support. The length of the main section 1220 can be determined according to actual needs. Alternatively, the projection of the profile of the outer wall of the main section 1220 in a plane parallel to the axis of the assistive component 1200 may be a curve as long as the main section 1220 can be abut against the inner wall of the inferior vena cava after expanding and the assistive component 1200 can be placed to be coaxially with the inferior vena cava.

In some embodiments, the first transition section 1210 and the second transition section 1230 are radially symmetric, and are arranged at both axial ends of the main section 1220. In other embodiments, the length of the first transition section 1210 is less than the length of the second transition section 1230, and the first transition section is closer to the filter 2000, so as to keep the filter 2000 to be coaxial with the assistive component 1200 in the blood vessel.

Referring specifically to FIG. 3, in an exemplary embodiment, the filter 2000 includes a retrieval part 2100 and a plurality of filter rod groups. Each of the filter rod groups includes a plurality of filter rods 2210. The proximal-to-heart ends of the filter rods in all the filter rod groups are connected to the retrieval part 2100. The filter rods 2210 in different filter rod groups have different lengths. All the filter rods 2210 in a same filter rod group have a same length, and are arranged symmetrically around the axis of the filter 2000. The connection between the filter 2000 to the delivery component 1100 will be described later in conjunction with the usage scenario.

Optionally, the shortest filter rods 2210 is configured to be in line contact with the vessel wall, and an anchor is provided at each distal end of the rest of the filter rods. The anchor is configured to penetrate the vessel wall so that the rest of the filter rods 2210 are in point contact with the vessel wall.

In the specification, the filter 2000 includes four filter rod groups as an example. The four filter rod groups are the first, second, third and fourth filter groups. The first filter rod group includes six first filter rods 2210a, the second filter rod group includes two second filter rods 2210b, the third filter rod group includes two third filter rods 2210c, and the fourth filter rod group includes two fourth filter rods 2210d. The length of the first filter rods 2210a, the length of the second filter rods 2210b, the length of the third filter rods 2210c and the length of the fourth filter rods 2210d are increased in sequence. Thus, the first filter rods 2210a are configured to be in line contact with the vessel wall, and an anchor is formed on each of the second filter rods 2210b, the third filter rods 2210c, and the fourth filter rods 2210d. Those skilled in the art may understand that the length of each of the filter rods 2210 refers to the size of the filter rod 2210 in the axial direction when it is in a compressed configuration.

Optionally, each of the first filter rods 2210a includes a first part 2211a, second part 2212a and third part 2213a successively connected with one another from the proximal-to-heart end to the distal-to-heart end. The distance between the first part 2211a and the axis of the filter 2000 gradually increases in a direction from the proximal-to-heart end to the distal-to-heart end, and the distance between the third part 2213a and the axis of the filter 2000 is greater than the distance between the first part 2211a to the axis of the filter 2000. The second part 2212a is actually a transition so as avoiding the first filter rod 2210a from forming a sharp corner. The second filter rods 2210b, the third filter rods 2210c and the fourth filter rods 2210d are all straight. The distance between the filter rods and the axis of the filter 2000 gradually increases in a direction from the proximal-to-heart end to the distal-to-heart end.

The anchors include first anchors 2310 and second anchors 2320. The first anchors 2310 and the second anchors 2320 are formed on the different filter rods 2210. When the first and second anchors penetrate the vessel wall, the first anchors 2310 are configured to prevent the filter 2000 from moving in a direction from the proximal-to-heart end to the distal-to-heart end, and the second anchors 2320 are configured to prevent the filter 2000 from moving in a direction from the distal-to-heart end to the proximal-to-heart end so that the filter 2000 is fixed in a predetermined position for effectively intercepting thrombus. In this embodiment, the first anchors 2310 may each be straight, the proximal-to-heart end of each of the first anchors 2310 is connected to the distal-to-heart end of the corresponding one of the filter rods 2210, and the distal-to-heart end of each of the first anchors 2310 is a free end. The second anchors 2320 each serve as a barb, and the free end of each of the second anchors 2320 is disposed toward the proximal-to-heart end of the filter 2000.

Generally, the filter rod 2210 on which the second anchor 2320 is formed is shorter than the filter rod 2210 on which the first anchor 2310 is formed. That is, in this embodiment, the first anchor 2310 is formed on the fourth filter rod 2210d, the second anchor 2320 is formed on the second filter rod 2210b, and either the second anchor 2320 (shown in FIG. 3) or the first anchor (not shown) is formed on the third filter rod 2210c. Further, a puncture-proof member 2410 is formed on at least some of, preferably, all of the filter rods 2210 on which the anchors are formed. The puncture-proof member 2410 is configured to prevent the anchor from piercing the vessel wall. Those skilled in the art may understand that the shape and size of the puncture-proof member 2410 may be changed for this purpose.

It should be noted that although the filter 2000 shown in FIG. 3 is presented as an example, it is understood that the filter in the prior art may also possible to be combined with the delivery mechanism 1000 to constitute the medical device.

Next, preferred structures of the medical device will be described in conjunction with the usage scenario thereof. Hereinafter, the “distal-to-heart end” and “proximal-to-heart end” mentioned in this specification are in terms of the positional relationship with the heart of the patient after the filter 2000 is implanted. Generally, the “distal-to-heart end” refers to the end of the filter 2000 farther away from the heart, and the “proximal-to-heart end” refers to the end of the filter 2000 closer to the heart. The terms “distal end” and “proximal end” are the relative orientation, position, or direction of the elements or actions in the delivery mechanism 1000 from the perspective of the operator using the medical device, the “distal end” refers to the end of the delivery mechanism 1000 that first enters the body of the patient, and the “proximal end” refers to the end closer the user during the use of the of the delivery mechanism 1000.

FIG. 4 is a schematic diagram of the medical device whose distal end enter the inferior vena cava via the jugular vein, where the filter 2000 is located at the distal end of the assistive component 1200.

Referring to FIG. 4 in conjunction with FIG. 2, the delivery component 1100 includes a tube assembly 1110 and a backward release component. The tube assembly 1110 includes at least a first tube 1111. The distal structure of the backward release component is partially connected to the first tube 111, preferably to the distal end of the first tube 1111. The distal structure of the backward release component is arranged coaxially with the first tube 1111. The assistive component 1200 is sleeved over the first tube 1111 and is configured to at least partially move in the axial direction of the first tube 1111 to expand or shrink the assistive component 1200. The proximal-to-heart end (in particular the retrieval part 2100) of the filter 2000 is detachably connected to the distal structure of the backward release component. The arrangement of the backward release component makes it not only convenient to control the expansion of the assistive component 1200 during the release of the filter 2000, but also possible to release the filter 2000 step by step (which means to release the filter rods and then disconnect the backward release component from the retrieval part 2100), thereby avoiding a displacement of the filter 2000 from a predetermined position resulting from the release of the filter 2000 as a whole.

Further, the tube assembly 1110 further includes a second 1112 that is sleeved over the first tube 1111 and is movable in an axial direction relative to the first tube 1111. The distal end of the assistive component 1200 remains stationary relative to the first tube 1111, and the proximal end of the assistive component 1200 is connected to the distal end of the second tube 1112 and moves synchronously with the second tube 1112 so as to be movable in the axial direction relative to the first tube 1111. As a result, the assistive component 1200 can be expanded or shrunk radially.

The backward release component may include a limit sleeve 1121, a restraint member 1122, and a connector (not shown). The limit sleeve 1121 and the restraint member 1122 together constitute the distal structure. The limit sleeve 1121 may be a cylindrical hollow tube connected to the first tube 1111 (in particular, to the distal end of the first tube 1111) and maintained axially stationary to the first tube 1111. The limit sleeve 1121 is in communication with the first tube 1111. The restraint member 1122 is configured to be inside the limit sleeve 1121 and is axially movable relative to the limit sleeve 1121. The connector is inserted in the first tube 1111 and is axially movable relative to the first tube 1111, and the distal end of the connector is connected to the restraint member 1122. The restraint member 1122 is engaged with the limit sleeve 1121 and connected to the retrieval part 2100 of the filter 2000 when the restraint member 1122 is at least partially located inside the limit sleeve 1121. When the restraint member 1122 moves relative to the limit sleeve 1121 and at least partially protrudes from the distal end of the limit sleeve 1121 to disengage from the limit sleeve 1121, the backward release component is disconnected from the retrieval part 2100.

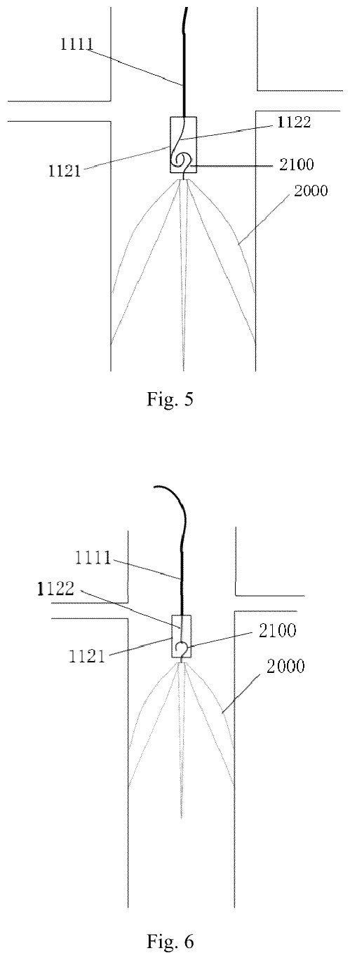

In an optional implementation, referring to FIG. 5, the retrieval part 2100 is a retrieval hook. The restraint member 1122 is an elastic member, for example has self-expanding structure. When the restraint member 1122 is located inside the limit sleeve 1121, the restraint member 1122 is bent to form as a hook for hooking the retrieval hook, and the limit sleeve 1121 applies radial pressure to the restraint member 1122 to maintain the restraint member 1122 in the shape of the hook so that the backward release component remains to be connected to the filter 2000. When the restraint member 1122 at least partially protrudes from the limit sleeve 1121 and the radial pressure applied to the restraint member 1122 by the limit sleeve 1121 is removed, the restraint member 1122 is returned to a non-hooklike shape, and the backward release component releases the filter 2000 so that they are disconnected from each other. The self-expanding structure here refers to the structure itself has good resilience, which can deform when subjected to pressure, and once the pressure is cancelled, the structure returns to its original shape under the action of its own resilience. Generally, the self-expanding structure is made of a shape memory alloy such as nickel-titanium alloy.

Optionally, in another embodiment, as shown in FIG. 6, the retrieval part 2100 may be a retrieval hook with a connection hole thereon. The restraint member 1122 is an elastic member, for example has a self-expanding structure. When the restraint member 1122 is located inside the limit sleeve 1121, the restraint member 1122 is bent to serve as a hook that partially extends into the connection hole to hook the retrieval hook.

Referring back to FIG. 2, the delivery component 1100 further comprises a handle 1130 provided with a first drive assembly 1140 and a second drive assembly. The handle 1130 is connected to the proximal end of the connector, the first drive assembly 1140 is connected to the proximal end of the second tube 1112 so as to drive the second tube 1112 to move axially relative to the first tube 1111. The second drive assembly is connected to the proximal end of the first tube 1111 so as to drive the first tube 1111 to move axially relative to the connector.

Referring further to FIG. 2, the handle 1130 has a slot 1131 extending along a axial direction of the handle 1130. The first drive assembly 1140 includes a first slider 1141 and a manipulator 1142. The first slider 1141 is disposed within the handle 1130 and movable along an axial direction of the handle 1130, and the first slider 1141 is connected to a proximal end of the second tube 1112. The manipulator 1142 movably disposed at the slot 1131. The manipulator 1142 has one end extending into the handle 1130 to be connected to the slider 1141, and the other end visible from the slot 1131. The manipulator 1142 is configured to be moved along the slot 1131 by an external force so as to drive the first slider 1141 to drive the second tube 1112 to move along the axial direction of the first tube 1111, thereby driving the proximal end of the assistive component 1200 in the axial direction of the first tube 1111 to expand or shrink the assistive component 1200. The second drive assembly is disposed proximally to the first drive assembly 1140. The second drive assembly includes a knob 1151 and a transmission (not shown) connected to the knob 1151. The first tube 1111 extends out of the proximal end of the second tube 1112 to be connected with the transmission, so that the second drive assembly may drive the first tube 1111 to move in an axial direction of the connector. The specific structure of the transmission may be configured with reference to the prior art.

Further, the delivery mechanism 1000 further includes a storage tube 1300 configured to compress the filter 2000. When the medical device is practically used, the retrieval part 2100 of the filter 2000 is first detachably connected to the distal structure of the backward release component, and the filter 2000 is then compressed by the storage tube 1300. Surgical operations can then be performed as follows:

First, the jugular vein was punctured, and a guidewire was introduced.

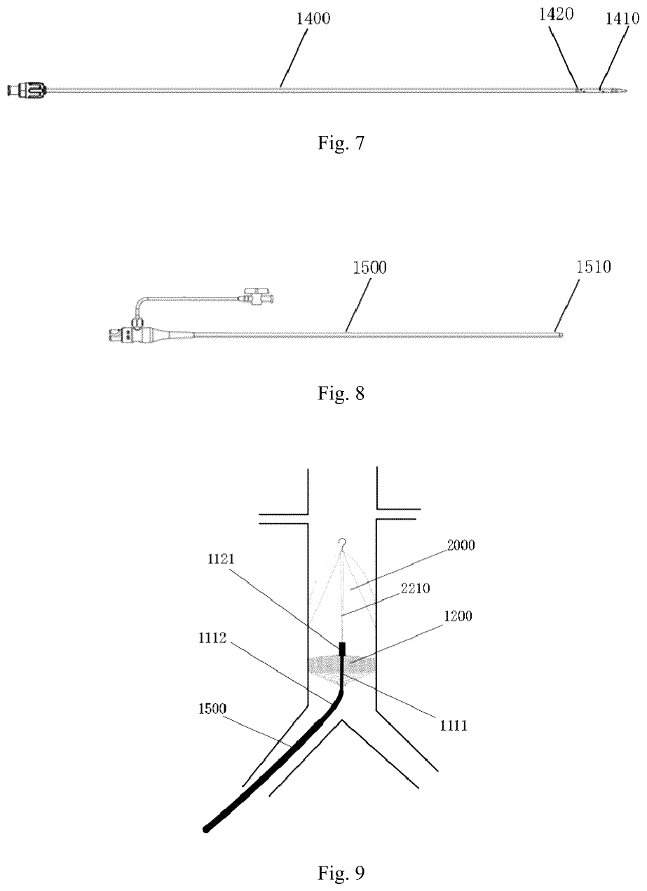

Next, an expander 1400 (shown in FIG. 7) travels into the sheath 1500 (shown in FIG. 8), and the distal ends of the expander 1400 and the sheath 1500 are delivered to the inferior vena cava through the jugular vein, and the radiographic hole 1410 of the expander 1400 is radiographed under an X-ray machine so as to determine the size of the vessel and the location of the filter 2000 (i.e., check the predetermined position), and then the distal end of the sheath 1500 is pushed to the predetermined position, and the expander 1400 is removed from the body.

Next, the distal end of the delivery mechanism 1000 carrying the filter 2000 is introduced to the predetermined position along the sheath 1500. It is understood that the assistive component 1200 is always shrunk during delivery. Accordingly, the filter 2000 has now been detached from the storage tube 1300 and is pushed to the distal edge of the sheath 1500.

Next, the sheath 1500 is withdrawn (i.e., the sheath is moved in a direction from the distal end to the proximal end) to radially expand the filter rods 2210 of the filter 2000, and the retrieval part 2100 of the filter 2000 is detachably connected to the backward release component.

Next, the sheath 1500 is further withdrawn until the assistive component 1200 is exposed, and the second tube 1112 is driven to move in a direction from the proximal end to the distal end by the first drive assembly 1140 so that expand the assistive component 1200 is expanded radially. The movement of the second tube 1112 can be adjusted according to the size of the vessel at the predetermined position so that the assistive component 1200 can be expanded to a size suitable for effectively supporting the vessel and being coaxially with the vessel. Since the filter 2000 is arranged coaxially with the tube assembly 1110 and the assistive component 1200, during the expansion of the assistive component 1200 for becoming coaxial with the vessel, the form of the filter 2000 is adjusted accordingly so that the filter 2000 is coaxial with the vessel.

Next, the first tube 1111 is driven to be moved in a direction from the distal end to the proximal end by the second drive assembly, and the limit sleeve 1121 is driven to move toward the proximal end. Since the connector remains stationary, the limit sleeve 1121 moves toward the proximal end relative to the restraint member 1122, so that the restraint member 1122 at least partially protrudes from the distal end of the limit sleeve 1121 until the filter 2000 is fully released.

The delivery mechanism 1000 is then withdrawn, the process of which is described as follows. The first tube 1111 drives the limit sleeve 1121 to move distally, the restraint member 1122 is received in the limit sleeve 1121, and the second tube 1112 is driven to move proximally by the first drive assembly so that the assistive component 1200 is shrunk radially. The sheath 1500 is then pushed distally until the distal end of the delivery mechanism 1000 (i.e., the limit sleeve 1121) enters the sheath 1500. Finally, the sheath 1500 and the delivery mechanism 1000 are completely withdrawn from the body.

It should be noted that the expander 1400 and the sheath 1500 used in the above shrinking process are the expander and sheath in the prior art. Therefore, the embodiments of the present invention do not describe their structures in details. Moreover, the structure and usage method of the storage tube 1300 also belong to prior art, which are not repeated here.

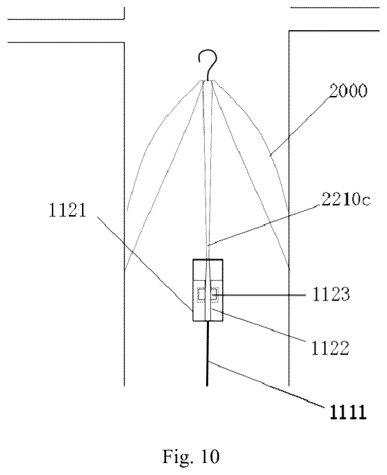

FIG. 10 is a schematic diagram showing that the filter 2000 is delivered by the delivery mechanism 1000 to a predetermined position in the inferior vena cava through the femoral vein. In FIG. 10, the assistive component 1200 is located at the distal end of the filter 2000. Referring to FIG. 10, the structure of the delivery mechanism 1000 in this usage scenario is identical to the structure of the delivery mechanism 1000 shown in FIG. 5, but the connection between the delivery component 1100 and the filter 2000 is different than that in FIG. 5.

Referring to FIGS. 3, 9 and 10, the first filter rod 2210a of the filter 2000 is compressed in the sheath 1500, and the rest of the filter rods 2210 are configured to be detachably connected to the backward release component. Specifically, all the distal-to-heart end of the second filter rods 2210b, the third filter rods 2210c and the fourth filter rods 220d are extended into the limit sleeve 1121, and the distal ends of the fourth filter rods 2210d which are the longest of the filter rods are connected to the restraint member 1122 and the limit sleeve 1121 by the anchors and/or the puncture-proof members 2410.

Optionally, with particular reference to FIG. 10, a second slider serves as the restraint member 1122, and the restraint member 1122 has a side wall defined therein with grooves 1123 shaped to match the first anchors 2310 and/or the puncture-proof members 2410 on the fourth filter rods 2210d. When the second slider is located at least partially within the limit sleeve 1121, the groove 1123 and the limit sleeve 1121 together define a space where the first anchors 2310 and/or puncture-proof members 2410 are restrained so that the filter 2000 is connected to the backward release component. When the restraint member 1122 moves axially relative to the limit sleeve 1121, the groove 1123 is at least partially exposed from the limit sleeve 1121, and the first anchor 2310 and/or the puncture-proof members 2410 are no longer restrained, and finally the filter 2000 is released.

Thus, when the distal end of the medical device is delivered to a predetermined position in the inferior vena cava, the operator first withdraws the sheath 1500 until the assistive component 1200 is exposed, and the first filter rods 2210a of the filter 2000 are released. The operator then moves the second tube 1112 distally so that the assistive component 1200 is expanded radially to be coaxially supported on the inner wall of the vessel, then moves the first tube 1111 proximally to successively release the second filter rods 2210b, the third filter rods 2210c, and the fourth filter rods 2210d. The filter 2000 is gradually released, thereby avoiding a displacement of the filter 2000 while avoiding the winding between the filter rods 2210, and ensures that all the filter rods 2210 are evenly distributed on the inner wall of the vessel, effectively covering the vessels and maintained to be centrally, improving the efficiency for thrombus interception, and facilitating the subsequent retrieval of the filter 2000.

It should be noted that, in the embodiments of the present invention, the tube assembly 1110 and the backward release component may be made of a polymer or metal material, and the second tube 1112 should have good flexibility which is good for adjusting the form of the filter 2000 so that the filter 2000 can be positioned centrally when the assistive component 1200 expands in the vessel. The sheath 1500 and the expander 1400 should have good flexibility and may be made of a polymer material such as HDPE or PA. A first visualization element 1510 should be provided at the distal end of the sheath 1500. A second visualization element 1420 and a radiographic hole 1410 should be provided at the distal end of the expander 1400. The material of the first visualization element 1510 and the second visualization element 1420 may be tantalum, platinum tungsten alloy or platinum iridium alloy or the like.

It may be understood that in an alternative embodiment, the first tube is provided with a perfusion channel, and a balloon (not shown) may serve as the assistive component. The balloon is sleeved over the first tube and connected with the perfusion channel so as to inject an agent into the balloon to expand the balloon. According to the actual use scenario, the balloon may be located proximal or distal to the limit sleeve of the backward release component. Further, the second tube and the first drive assembly may be omitted when using the balloon as the assistive component.

Although the present invention is disclosed as above, it is not limited thereto. Those skilled in the art may make various modifications and variations of the invention without departing from the spirit and scope of the invention. Thus, if these modifications and variations of the invention fall within the scope of the claims and the equivalent technology, the invention is also intended to include these modifications and variations.

What is claimed is:

1. A medical device, comprising a delivery mechanism and an implant, wherein the delivery mechanism comprises a delivery component and an assistive component that is connected to the delivery component and configured to be expanded or shrunk in a radial direction of the delivery component, and the implant is detachably connected to the delivery component;
wherein the delivery mechanism is configured to deliver the implant to a predetermined position in a target lumen and release the implant, and the assistive component is radially expanded during the release of the implant so as to release the implant with a predetermined form in the predetermined position.

2. The medical device of claim 1, wherein the delivery component comprises a tube assembly and a backward release component, the tube assembly comprising at least a first tube, the backward release component having a distal structure that is partially connected to the first tube and coaxially to the first tube, the assistive component sleeved over the first tube and proximal to the distal structure, the implant detachably connected to the distal structure of the backward release component.

3. The medical device of claim 2, wherein the assistive component comprises a first transition section, a main section and a second transition section which are axially connected successively, and an outer diameter of the first transition section and an outer diameter of the second transition section decrease in directions away from the main section;
wherein the assistive component is configured to at least partially movable in an axial direction of the first tube to radially expand or shrink the assistive component.

4. The medical device of claim 3, wherein an outer diameter of at least some segments of the main section is greater than or equal to a maximum outer diameter of the first transition section and the second transition section.

5. The medical device of claim 3, wherein the tube assembly further comprises a second tube that is sleeved over the first body and axially movable relative to the first tube and the distal structure;
wherein the assistive component has a distal end remained stationary relative to the first tube, and a proximal end that is connected to a distal end of the second tube and movable synchronously with the second tube.

6. The medical device of claim 5, wherein the delivery component further comprises a handle provided with a first drive assembly that is connected to a proximal end of the second tube and configured to drive the second tube to move axially relative to the first tube.

7. The medical device of claim 3, wherein the first transition section and the second transition section are symmetrically disposed at two axial ends of the main section; or
wherein a length of the first transition section is less than a length of the second transition section, and the first transition section is closer to the implant.

8. The medical device of claim 2, wherein the assistive component is a balloon that is connected to and in communication with a perfusion channel defined in the first tube, and the perfusion channel is configured to fill an agent into the balloon.

9. The medical device of claim 2, wherein the backward release component comprises a limit sleeve, a restraint member and a connector, wherein the limit sleeve and the restraint member constitute the distal structure of the backward release component, the limit sleeve is connected to and in communication with the first tube, the limit sleeve is axially stationary relative to the first tube; the restraint member is configured to be received in the limit sleeve; the connector is inserted into the the first tube and movable relative to the first tube, and a distal end of the connector is connected with the restraint member; wherein the restraint member is engaged with the limit sleeve and the restraint member is detachably connected to the implant when the restraint member is at least partially located inside the limit sleeve, and the backward release component is disconnected from the implant when the restraint member is at least partially exposed from a distal end of the limit sleeve and the restraint member is disengaged with the limit sleeve, resulting from a movement of the first tube relative to the connector.

10. The medical device of claim 9, wherein the delivery component further comprises a handle provided with a second drive assembly, the handle connected to a proximal end of the connector, the second drive assembly connected to a proximal end of the first tube and configured to drive the first tube to move axially relative to the connector.

11. The medical device of claim 9, wherein the implant comprises a plurality of filter rod groups and a retrieval part, each of the filter rod groups comprising a plurality of filter rods, proximal-to-heart ends of the filter rods in all the filter rod groups connected to the retrieval part; wherein the filter rods in different ones of the filter rod groups have different lengths, and all the filter rods in a same one of the filter rod groups have a same length and are arranged symmetrically around an axis of the implant;
wherein the retrieval part is configured to detachably connect to the backward release component; or
the medical device further comprises a sheath, shortest ones of the filter rods configured to be compressed in the sheath, distal-to-heart ends of rest of the filter rods configured to be inserted into the limit sleeve, distal-to-heart ends of longest ones of the filter rods configured to be connected to the restraint member and the limit sleeve.

12. The medical device of claim 9, wherein the restraint member is a slider having a side wall defined therein with a groove;
wherein the groove and an inner surface of the limit sleeve together limit a position of a proximal-to-heart end or a distal-to-heart end of the implant so as to connect the implant with the backward release component when the slider is at least partially received in the limit sleeve, and the backward release component is disconnected from the implant when the slider moves axially relative to the limit sleeve to expose the groove from the limit sleeve.

13. The medical device of claim 9, wherein the restraint member is an elastic member; when the elastic member is received in the limit sleeve, the limit sleeve applies a radial pressure to the elastic member to shape the elastic member as a hook for hooking the implant; when the elastic member at least partially protrudes from the limit sleeve, the radial pressure applied by the limit sleeve to the elastic member is removed, and the elastic member returns to a form that does not have the hook to release the implant.
